(19) United States
(12) Patent Application Publication
(10) Pub. No.: US 2006/0188391 A1
(43) Pub. Date: Aug. 24, 2006

(54) Chlorohydrin removal method

(76) Inventors: Kenneth F. Cherry, Toledo, OH (US); Jill R. Cherry, Toledo, OH (US)

Correspondence Address: Kenneth F Cherry, 1533 Eastgate, Toledo, OH, 43614, US

(21) Appl. No.: 11/060,159

(22) Filed: Feb. 18, 2005

(51) Int. Cl. A61L 2/20

(52) U.S. Cl. 422/34; 422/30; 423/462

(57) ABSTRACT

In the use of ethylene oxide as a sterilizing agent, toxic chemicals known as chlorohydrins may be formed in the sterilized materials. Chlorohydrins are difficult to remove using the normal pressure vacuum air purge cycles specified in the ethylene oxide sterilization process. Introduction of ammonia vapors in one or more of the pressure vacuum purge cycles provides a means to reduce chlorohydrin concentration in the sterilized materials.

### CROSS REFERENCE TO RELATED APPLICATIONS

None

### STATEMENT OF FEDERALLY SPONSORED RESEARCH

None

### REFERENCE TO A MICROFICHE APPENDIX

None

### BACKGROUND OF THE INVENTION

Ethylene oxide is used as a sterilizing agent as an alternative to methods such as high temperature steam, sterilization and radiation sterilization for the destruction of microorganisms. These and other methods are somewhat interchangeable and used to destroy bacteria, viruses, and fungi. This destruction of microorganisms reduces the chance of infection of persons in contact with the sterilized material and degradation of materials which are to be stored. In addition, microorganism destruction is advantageous in other well-known applications.

The use of ethylene oxide as a sterilizing agent may produce chlorohydrins as a chemical by-product of the procedure. Chlorohydrins are known to be toxic compounds, which may be absorbed through the skin. In addition, chlorohydrins are difficult to remove from materials that have been sterilized.

This invention provides a method to destroy chlorohydrins after their formation in ethylene oxide sterilization processes through the use of ammonia as a reactant chemical.

### BRIEF SUMMARY OF THE INVENTION

Ethylene oxide is used for the sterilization of materials to kill or deactivate spores, viruses, bacteria as well as any live organisms of other classifications. The ethylene oxide sterilization step occurs in a pressure-vacuum chamber. After sterilization, it is sometimes found that materials have chlorohydrin formed as a result of the sterilization process; this is especially true in paper and other bleached products. The chlorohydrin is not removed effectively in the usual pressure-vacuum purging steps of the ethylene oxide sterilization process. The introduction of ammonia into one or more of the purge cycle steps will reduce or eliminate the chlorohydrin concentration in the sterilized materials. This reduction of chlorohydrins is beneficial because chlorohydrin is a toxic substance, which may be absorbed through the skin or directly into tissues and cause toxic effects in people or animals. Incorporation of the ammonia contact cycle and the usual ethylene oxide sterilization process can be accomplished with minor modifications of the usual equipment used for ethylene oxide sterilization. Other handling of the sterilized materials including air purge cycles for off-gassing of volatiles is relatively unchanged.

### BRIEF DESCRIPTION OF THE DRAWING

FIG. 1 is a flow diagram of the process.

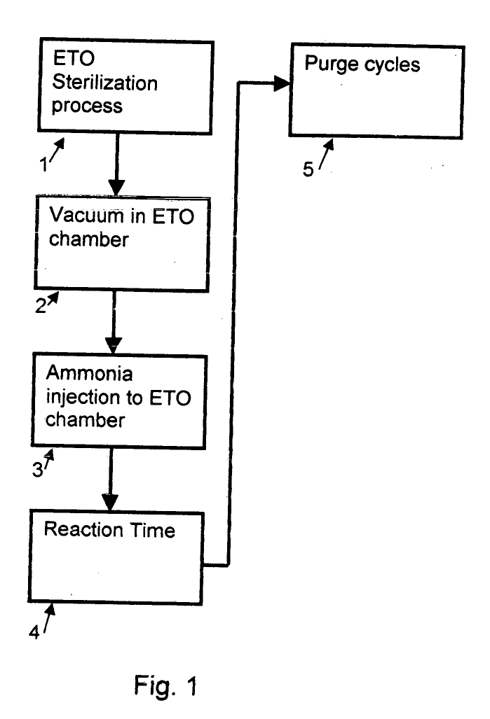

Step 1 is the normal process of ethylene oxide applied in a chamber.

Step 2 is the vacuum steps used to remove ethylene oxide after sterilization is complete.

Step 3 is the introduction of ammonia into the chamber in the same basic procedure as was used for the process noted in step 1.

Step 4 is allowing time for the reaction to occur.

Step 5 is one or more purge cycles to remove ammonia from the treated materials.

### DETAILED DESCRIPTION OF THE INVENTION

Toxic chemicals called chlorohydrins may be formed during the process of using ethylene oxide as a sterilizing agent. Chlorohydrins will be absorbed through the skin or into tissue in contact with the sterilized materials. Introduction of chlorohydrins into a human or animal biological system may have significant toxic effects, even at low concentrations.

The sterilization procedure using ethylene oxide, often referred to as ETO, usually includes putting the material to be sterilized into a chamber. Air is evacuated from the chamber and in a subsequent step ethylene oxide is introduced to the chamber and permeates the material to be sterilized. Introduction of an inert gas and moisture are also often part of the process. After the permeation, a period of time is allowed for the ethylene oxide to interact with and destroy microorganisms. The removal of the ethylene oxide is accomplished by subsequent vacuum and pressure cycles where in the ethylene oxide is removed and purged with air. In addition, it is common to have a period of time for additional ethylene oxide evaporation from the sterilized materials in a separate chamber with controlled temperature and humidity.

Testing for biological activity and the presence of chlorohydrin is a step, which may be performed after the sterilization process is considered complete. If chlorohydrin is found additional vacuum and pressure cycles using air may reduce some of the chlorohydrin concentration. This reduction is often inadequate, especially in paper products and materials, which have been bleached with chlorine or that contain some salts. It is postulated that the residual chlorine compounds and bleached materials react with the ethylene oxide to produce the chlorohydrin. Other undesirable byproducts such as ethylene glycol may also be formed.

Removal and destruction of chlorohydrin in the sterilized materials may be accomplished by the introduction of ammonia vapors. The injection of ammonia may also reduce the residual ethylene oxide and ethylene glycol. For the purposes of this patent residual ethylene oxide, chlorohydrins and ethylene glycol type compounds are all considered residual contamination. This introduction is preferably accomplished during one or more of the pressure and vacuum cycles in the chamber in a manner similar used for the removal of ethylene oxide from the sterilized materials.

Reaction time to reduce the chlorohydrin levels is dependent on temperature and pressure. It is suggested starting point for the removal of chlorohydrins from materials such as textbooks that have been sterilized includes two pressure vacuum cycles with air after the sterilization with ethylene oxide followed by one or more pressure vacuum cycles with ammonia. The time of contact with the ammonia and materials that have been sterilized is suggested to begin at one-hour contact time; however, shorter periods may be appropriate depending on the concentration of chlorohydrins and the material in which they are imbedded. An amount of ammonia equal to the amount of ethylene oxide is suggested as a starting point. Adjustments in time, concentrations and purge cycles may be required.

After chlorohydrin deactivation by the ammonia, one or more pressure vacuum cycles with air to remove the ammonia odor is suggested. In addition, an off-gassing period in a separate chamber is usually appropriate to insure that all volatiles including possible residual ethylene oxide and ammonia have time to evaporate. The final evaporation step may take a day or two at temperatures approximating 100 degrees Fahrenheit. The temperatures and times for all of the above steps may be adjusted or optimized as required to obtain efficiency with a specific material to be sterilized.

Experimental results have shown that higher concentrations of ammonia vapor are more effective than lower concentrations. The ammonia may be injected into the pressure chamber in using several methods.

In one preferred embodiment of the invention, the ammonia is injected into the airline that would otherwise be used for providing air for the purge cycle. Injection of liquid anhydrous ammonia has been found to be one effect means of introducing the ammonia vapors into the chamber. Other methods, such as use of more dilute ammonia materials or the introduction of gaseous ammonia are alternatives that are equivalent.

I claim:

1. A method to reduce chlorohydrin concentration produced during ethylene oxide sterilization comprising an ammonia injection step to provide ammonia contact with the sterilized materials resulting in a reduction in the chlorohydrin concentration, in the sterilized materials.

2. The invention of claim 1 where in the ammonia introducing step is incorporated with the air pressure and vacuum steps specified in the commonly used ethylene oxide sterilization procedures.

3. Invention of claim 1 where the ammonia is introduced as liquid anhydrous ammonia into the pressure chamber.

4. The invention of claim 1 where ammonia is introduced into the pressure vacuum chamber, as a gaseous material in concentrated form.

5. The invention of claim 1 where in the method is used to remove chlorohydrin from paper materials.

6. The use of sterilized materials produced by the invention of claim 1.

7. The use of ammonia injection after ethylene oxide sterilization to reduce the amount of residual contamination in the sterilized material.
